(19) United States
(12) Patent Application Publication
(10) Pub. No.: US 2010/0022996 A1
(43) Pub. Date: Jan. 28, 2010

(54) METHOD AND SYSTEM FOR CREATING A BUBBLE SHIELD FOR LASER LENS PROCEDURES

(76) Inventors: Rudolph W. Frey, Maitland, FL (US); Steven E. Bott, Oviedo, FL (US)

Correspondence Address: BRINKS HOFER GILSON & LIONE, P.O. BOX 10395, CHICAGO, IL, 60610, US

(21) Appl. No.: 12/509,211

(22) Filed: Jul. 24, 2009

Related U.S. Application Data
(60) Provisional application No. 61/083,847, filed on Jul. 25, 2008.

(51) Int. Cl. A61F 9/008; A61B 18/20

(52) U.S. Cl. 606/6

(57) ABSTRACT

There is provided a system and method for creating a layer of bubbles in the posterior portion of the lens of the eye. This layer of bubbles in the posterior portion of the lens functions as a shield protection structures of the eye that are located posteriorly from the lens from being damaged or injured by the use of the laser in subsequent activities upon the lens, such as the performance of a laser assisted capsulotomy. Thus, the bubble shield reduces the risk to posterior structures during laser procedures on the lens of the eye.

This application claims the benefit of priority of provisional application Ser. No. 61/083,847 filed Jul. 25, 2008. The present invention relates to methods and systems for providing a shield of bubbles in the lens of an eye during laser lens surgery to protect the retina and other structures of the eye that are posterior to the lens from detrimental effects of the laser.

### BACKGROUND OF THE INVENTION

In general, cataracts are areas of opacification of the ocular lens which are sufficient to interfere with vision. Other conditions for which the present invention may be useful, include, but are not limited to, the opacification of the ocular lens and the extraction of a clear lens. Cataracts, or the condition when the natural crystalline lens becomes opaque and clouds vision, occur in millions of people per year and are treated effectively with surgical techniques, such as ultrasonic phacoemulsification pioneered by Kelman 30 years ago. Although the techniques have been refined over the years, safety concerns over ocular trauma arise, especially damage to the corneal endothelium from the ultrasonic energy required to break up a hardened cataract, particularly for those with a compromised corneal endothelium, such as those with Fuchs Dystrophy.

Thus the use of lasers in the treatment of cataracts has been suggested. As with the use of ultrasound care must be taken to make certain that when the laser is used to cut or section the lens, the laser does not inadvertently strike and disrupt other structures in the eye that are located posterior to the lens.

### SUMMARY

Thus the present invention provides for the creation of a bubble shield within the lens art the commencement of lens surgery. This bubble shield would then prevent the laser beam from penetrating posterior to the bubble shield with sufficient power to disrupt or otherwise damage structures of the eye that are posterior to the shield. This shield will guard against, physician and apparatus errors that could cause the laser's energy to impinge on structures that are posterior to the lens.

The present invention may be employed by or with the systems for delivering laser beams to the lens of the eye, such as those disclosed in published applications US 2007/0173794 A1, US 2007/0173795 A1, US 2007/0185475 A1, WO 2007/084694 A2 (now U.S. Ser. No. 12/217,295), WO 2007/084627 A2 (now U.S. Ser. No. 12/217,285), the disclosure of which are incorporated herein by reference, and in co-pending applications lawyer docket no. 12212/51 and no. 12212/46 to Frey et al. filed on the same day as the present application, the disclosure of which is incorporated herein by reference.

Provided herein are embodiments of the present invention. There is provided a system for creating a shielding layer in a lens of an eye, the system having a laser for producing a laser beam, an optical path having focusing and scanning means for delivering the laser to the lens of the eye, and a control system for directing the laser beam in a shot pattern to a portion of the posterior section of the lens of the eye to create a bubble shield in the lens.

There is further provided a system for creating a shielding layer in a lens of an eye, the system having a laser that is capable of providing laser beam pulses to the lens of the eye at a first energy and first average power, the laser is also capable of providing laser beam pulses to the lens of the eye at a second energy and second average power, or a second laser may be used at a second energy and second average power, wherein the second average power is greater than or equal to the first average power or the second energy is greater than the first energy. The first energy being sufficient to create a bubble in the lens of the eye with each laser pulse when the laser beam is directed into the lens and a control system for directing the laser beam at the first energy to the lens of the eye, the shot pattern provides for the placement of a series of shots primarily to a portion of the posterior section of the lens of the eye to create a bubble shield in the lens. Upon completion of the formation of the bubble layer with the laser of the first energy, the laser of the second energy is capable of performing a subsequent activity on the lens of the eye such as a capsulotomy, and/or the sectioning and removal of the lens of the eye.

There is additionally provided a method of creating a bubble shield in a lens of an eye, the method having the steps of providing a laser beam in a laser shot pattern to the posterior portions of the lens of the eye, wherein the laser shot pattern has a shot density and the laser beam has an energy, wherein the shot density and the energy are such that the laser beam creates a continuous, or nearly continuous, layer of bubbles in the posterior portion of the lens of the eye.

One of ordinary skill in the art will recognize, based on the teachings set forth in these specifications and drawings, that there are various embodiments and implementations of these teachings to practice the present invention. Accordingly, the embodiments in this summary are not meant to limit these teachings in any way.

### BRIEF DESCRIPTION OF THE DRAWINGS

FIG. 1 is a schematic diagram of an eye showing the lens of the eye and a shot pattern for creating a bubble shield.

### DESCRIPTION OF THE DRAWINGS AND THE PREFERRED EMBODIMENTS

In general, the present invention relates to methods and systems for providing a bubble shield within the natural human crystalline lens to address, reduce and eliminate concerns regarding inadvertent damage to structures of the eye that could occur when using a laser to remove cataracts, opacifications in the lens, clear lens extraction, removal of natural lens material, replacement of that material with replacement material, and combinations of these. Thus, the present invention provides a system and method for delivering a laser to the posterior portion of the lens at a very low average power and energy level, at an energy level just above the point where bubble formation occurs in the lens. The laser, at this low energy and average power, is then applied to this posterior section of the lens to create a layer of bubbles within the lens. The average power and energy levels are chosen to be such that no damage is caused to structures in the eye posterior to the point at which the bubble shield is created.

This layer of bubbles functions as a shield during the rest of the laser procedure. Thus, if the laser's energy or average power is increased and/or a different laser of higher energy or average power is used on the lens, the layer of bubbles will prevent the higher energy laser beam from striking any structures posterior to the layer of bubbles with sufficient energy to damage those structures. Similarly, if for some reason a low energy laser, of about the same energy as the laser used to create the bubble layer is used, the bubbles will shield the posterior structures of the lens from damage from this low energy laser as well.

In general, a treatment laser for use in and on the lens should provide a beam that is of a wavelength that transmits through the cornea, aqueous and lens. The beam should be of a short pulse width, together with the energy and beam size, to produce photodisruption. Thus, as used herein, the term laser shot or shot refers to a laser beam pulse delivered to a location that results in photodisruption. As used herein, the term photodisruption essentially refers to the conversion of matter to a gas by the laser. In particular, wavelengths of about 300 nm to 2500 nm may be employed. Pulse widths from about 1 femtosecond to 100 picoseconds may be employed. Energies from about a 1 nanojoule to 1 millijoule may be employed. The pulse rate (also referred to as pulse repetition frequency (PRF) and pulses per second measured in Hertz) may be from about 1 KHz to several GHz. Generally, lower pulse rates correspond to higher pulse energy in commercial laser devices. A wide variety of laser types may be used to cause photodisruption of ocular tissues, dependent upon pulse width and energy density. Thus, examples of such lasers are disclosed in 2007/084694 A2 and WO 2007/084627A2, which are incorporated herein by reference. These and other similar lasers may be used a therapeutic lasers.

In general, delivery of laser shot patterns to the lens may be used for the removal and/or cutting of lens material. Three such primary activities are utilized, although other such activities may also be employed and the use of the present invention is not limited to being used in conjunction with these three activities. The first activity involves the anterior surface of the lens capsule and the use of the laser to perform a capsulotomy. The second activity involves the lens material that is contained within the capsule, i.e., the interior structures of the lens, such as the cortex and the nucleus and cataractous material contained therein. In this activity the laser is used alone, or in conjunction with other tools, to section the lens material into small parts so that the sectioned lens material can be aspirated from the capsule. The third activity involves the use of a lens replacement material, including but not limited to IOLs, within the lens capsule after interior structures of the lens have been removed. In all of these activities prior to performing the activity a layer of bubbles along the posterior side of the lens may be employed before the activity begins. In this way once the activity is begun the layer of bubbles will shield and protect the structures posterior to that layer from in advertent or accidental damage from the laser while the activity is being performed.

In creating the bubble layer it is preferred, although not required, that the lowest possible energy and average power to create bubble shield be used. The use of this low energy and low average power laser beam to create the bubble layer reduces the risk that structures posterior to the lens could be in advertently damaged during the formation of the bubble layer.

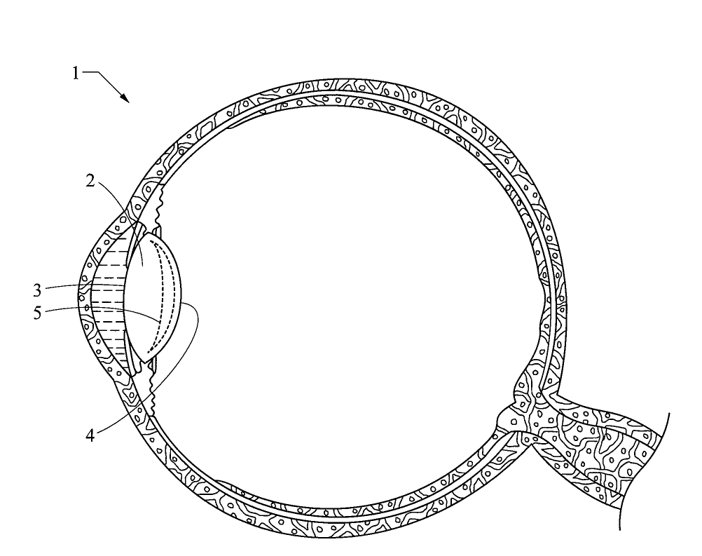

Turing then to FIG. 1 there is shown a representation of an eye 1 having a lens 2. The lens has an anterior side 3 and a posterior side 4. There is also shown a shot pattern 5. The shot pattern may be provided to the lens o f the eye along the posterior side of the lens for creating a bubble layer. The bubble layer should be of sufficient thickness, density and diameter to prevent the treatment laser used in later activities from penetrating the layer with sufficient energy to damage the underlying structures of the eye.

Thus is it contemplated that the bubble layer could be from about 0.1 mm to about 1.5 mm, preferably from about 0.25 mm to about 0.75 mm and more preferably about 0.5 mm anterior to the posterior capsule. It is further contemplated that the diameter of the bubble layer be large enough to shield the posterior section of the eye during the later activity, thus depending upon the shot pattern for the laser that is used in the later activity the diameter of the bubble layer could be larger or smaller. Thus, for example when the later activity is limited to a capsulotomy the diameter of the bubble shield would not be required to be much larger than the diameter of the capsulotomy, although for safety reasons larger diameter shields may still be desirable.

In the laser shot patterns provided herein for forming the bubble shield layer it is preferred that the laser shot patterns generally follow the shape of the lens and placement of individual shots with respect to adjacent shots in the pattern are sufficiently close enough to each other, such that when the pattern is complete a sufficiently continuous or nearly continuous layer of bubbles has been formed. Shot spacing of lesser or greater distances are contemplated herein and including overlap as necessary to obtain the desired results. The bubble shield may comprise several individual layers or sub-shields of bubbles. The shape and size of the bubble shield may be designed to provide protection for later treatments of various sizes and shapes, and the density of bubbles within the shield may provide more or less shielding over different portions of the shield. Moreover, the forgoing shot spacing considerations are interrelated to a lesser or greater extent and one of skill in the art will know how to evaluate these conditions based upon the teachings of the present disclosure to accomplish the objectives herein. Finally, it is contemplated that the placement of individual shots with respect to adjacent shots in the pattern may in general be such that they are as close as possible, typically limited by the size and time frame of photodisruption physics, which would include among other things gas bubble expansion of the previous shot. As used herein, the time frame of photodisruptive physics referrers to the effects that take place surrounding photodisruption, such as plasma formation and expansion, shock waive propagation, and gas bubble expansion and contraction.

What is claimed:

1. A system for creating a shielding layer in a lens of an eye, the system comprising:
a. a laser for producing a laser beam, and,
b. a control system for directing the laser beam in a shot pattern to a portion of the posterior section of the lens of the eye to create a bubble shield in the lens.

2. A system for creating a shielding layer in a lens of an eye, the system comprising:
a. a laser;
b. the laser capable of providing a laser beam to the lens of the eye at a first energy;
c. the laser capable of providing a laser beam to the lens of the eye at a second energy, wherein the second energy is greater than the first energy;
d. the first energy being sufficient to create a bubble in the lens of the eye when the laser beam is provided to the lens; and,
e. a control system for directing the laser beam at the first energy to the lens of the eye, the shot pattern provides for the placement of the shots primarily to a portion of the posterior section of the lens of the eye to create a bubble shield in the lens.

3. A method of creating a bubble shield in a lens of an eye, the method comprising:
a. providing a laser beam in a laser shot pattern to the posterior portions of the lens of the eye;
b. the laser shot pattern having a shot density; and,
c. the laser beam having an energy;
d. wherein the shot density and the energy are such that the laser beam creates an essentially continuous layer of bubbles in the posterior portion of the lens of the eye.
